(19) United States
(12) Patent Application Publication
(10) Pub. No.: US 2021/0010060 A1
(43) Pub. Date: Jan. 14, 2021

(54) Highly Multiplexed PCR with Bioinformatically Optimized Primers to Prepare Targeted Libraries for Next-Generation Sequencing

(71) Applicant: Aaron Zhang-Chen, San Diego, CA (US)

(72) Inventor: Aaron Zhang-Chen, San Diego, CA (US)

(73) Assignee: Genenius Genetics, San Diego, CA (US)

(21) Appl. No.: 16/915,622

(22) Filed: Jun. 29, 2020

Related U.S. Application Data
(60) Provisional application No. 62/869,942, filed on Jul. 2, 2019.
(60) Provisional application No. 62/876,635, filed on Jul. 20, 2019.

(51) Int. Cl. C12Q 1/686; C12Q 1/6869; C40B 40/06; C40B 60/14; C12Q 1/6853

(52) U.S. Cl. CPC C12Q 1/686; C12Q 1/6869; C12Q 2600/16; C40B 60/14; C12Q 1/6853; C40B 40/06

(57) ABSTRACT

Methods for obtaining libraries of multiple amplicons of target sequences with self-checking controls and sequences. Iterative bioinformatic methods for primer design with self-checking controls for optimized use of sequencing resources. Reagent cocktails for enrichment of target sequences.

### CROSS-REFERENCE TO RELATED APPLICATIONS

This application claims the benefit of U.S. provisional application 62/869,942, filed Jul. 2, 2019, entitled Highly Multiplexed PCR to Prepare Targeted Libraries for Next-Generation Sequencing, and U.S. provisional application 62/876,635, filed Jul. 20, 2019, entitled Bioinformatic Optimization of Primers for Highly Multiplexed PCR to Prepare Targeted Libraries, the contents of both of which are incorporated by reference herein.

### TECHNICAL FIELD

Amplification of nucleic acids for sequence determination. Primer sets for multiplex assays. Bioinformatic methods for optimizing primer sequences, grouping and amplicon balancing for amplification of target sequences.

### SUMMARY OF THE INVENTION

The present invention provides methods for obtaining libraries of multiple amplicons of target sequences to be sequenced. Multiple sets of tagged primers amplify different regions of the targets in separate groups of reactions. The initial amplification products can be pooled for efficient sequencing workflows and to yield multiple measurements of targets with self-checking barcode controls. The present invention provides iterative feedback methods for primer design, grouping, balancing, and optimized use of sequencing resources. The invention further provides reagent cocktails for enrichment of target sequences.

### BRIEF DESCRIPTION OF THE DRAWINGS

FIG. 1 illustrates a representative amplification scheme with a first and a second group of reactions (and optional other reactions) in a first polymerase chain reaction step (PCR 1). A double-stranded target sequence is shown. In the reaction of the first group, three primers are provided to amplify a first amplicon region of the target sequence (sometimes annotated as 1). The first amplicon may be visualized as the sequence from SpF_1 to SpR_1 (inclusive), on either strand of the target sequence.

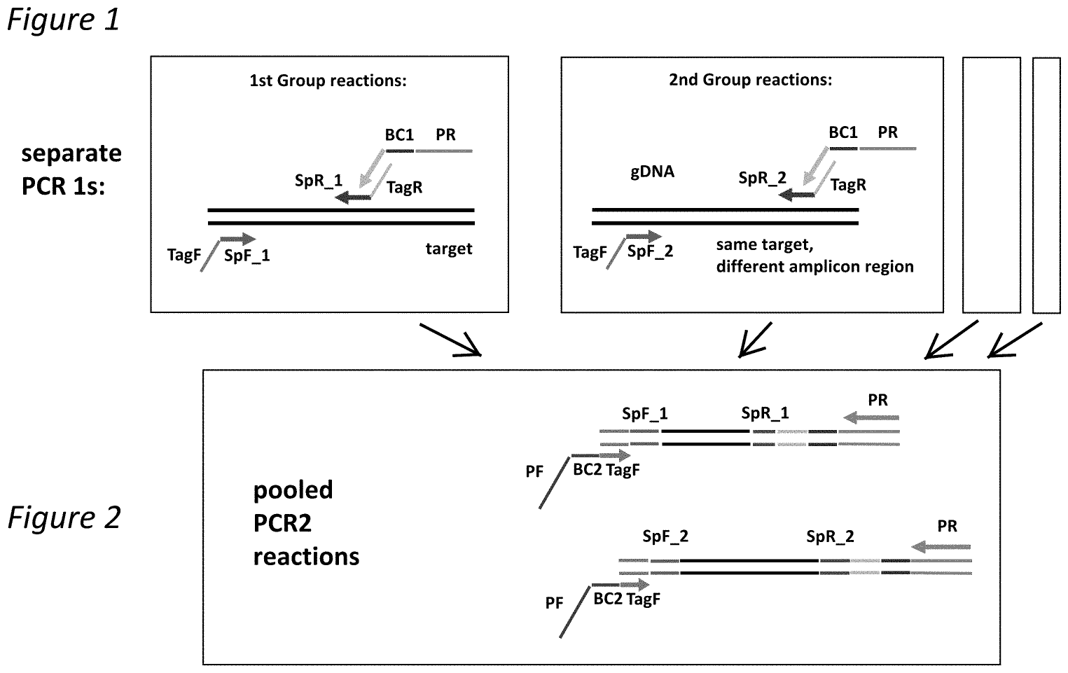

For convenience of discussion, the left side of the target sequence as illustrated is sometimes designated the “upstream” or “F” side and the right side is the “downstream” or “R” side. For example, in FIG. 1, the target sequence can be said to have an SpF portion on the left or upstream end and an SpR portion on the right or downstream end. These terms are relative and are not intended to be limiting as to the direction of gene transcription or translation, or the direction of amplification during PCR. Also, for convenience of illustration and discussion, sequences are labeled without regard to strand orientation (i. e., whether the sense or antisense strand is shown). The skilled artisan is able to design from such schematics how the amplification primers are to be in an orientation appropriate for a desired application, such as PCR, and with appropriate complementary or reverse-complementary sequences for cases in which a target strand or first amplified strand is available for hybridization.

As shown, a first primer has a forward tag sequence (TagF) and the upstream portion of the first amplicon (SpF_1). A second primer is provided, having a reverse tag sequence (TagR) and the downstream portion of the first amplicon (SpR of _1). An optional universal primer is also shown, having the reverse tag sequence (TagR) and other sequences as desired, such as an optional barcode (BC1) or PR sequence. These primers amplify the target sequence to generate first amplicons (i.e., from SpF_1 to SpR_1) as shown in FIG. 2.

A similar set of oligos are provided for the reactions of the second group, which amplify a different region of the same target sequence (i. e., from SpF_2 to SpR_2). These oligos include a first primer having the forward tag sequence (TagF) and the upstream portion of the (SpF_2); and a second primer having the reverse tag sequence (TagR) and the downstream portion of the region (SpR_2). The optional universal primer is also shown in this second group of reactions (TagR, BC1, PR). The amplicons resulting from the second group of reactions is shown as the amplicon containing the portion of the target sequence as shown from SpF_2 to SpR_2.

In FIG. 2, products of the first and second groups of reactions can be pooled. As an option, a supplemental reaction (PCR2) can be performed using a pair of supplemental amplifications primers. For example, one supplemental primer can have the sequence PF, an additional, optional barcode (BC2), and TagF. A second supplemental primer can have the sequence PR. One representative product of performing PCR2 can be represented as PF-BC2-TagF-SpF_1-(an intervening sequence of the target sequence)-SpR1-TagR-BC1-PR. Another representative product can be PF-BC2-TagF-SpF_2-(another intervening sequence of the target sequence)-SpR_2-TagR-BC1-PR. Such products of PCR2 can be analyzed further, such as by sequencing.

### DETAILED DESCRIPTION OF THE INVENTION

Conventional methods for targeted sequencing involve the amplification of known and variant sequences of interest from complex samples. PCR (polymerase chain reaction) and other amplification methods can be used to prepare libraries of amplicons for sequencing using commercially available workflows. However, the design of earlier methods can result in libraries having unintended or undesirable amplicons that are not representative of the sequences in the original sample. Earlier amplicon libraries can also suffer from unequal amplification when the sequences of interest are present in a potentially wide dynamic range. The more prevalent sequences that are often present in natural samples can take up the resources of amplification and sequencing reactions. The present invention provides methods for obtaining libraries of multiple amplicons of target sequences to be sequenced. Multiple sets of tagged primers are designed to amplify different regions of the targets in separate groups of reactions. The initial amplification products can then be pooled for efficient sequencing workflows and to yield multiple measurements of targets with self-checking controls.

The samples are typically from a biological organism, but can be from artificially created or environmental samples. Biological samples can be from living or dead animals, plants, yeast and other microorganisms, prokaryotes, or cell lines thereof. The samples can be crude samples, in the form of whole organisms or systems, tissue samples, cell samples, subcellular organelles, or samples that are cell-free, or viruses. Other examples include whole or fractionated blood samples, plasma, and serum.

The nucleic acids to be amplified can be from nucleic acid strands that are DNA, such as nuclear or mitochondrial DNA, or cDNA that is reverse-transcribed from RNA, such as mRNA, rRNA, tRNA, siRNAs, antisense RNAs, circular RNAs, or long noncoding RNAs, circular RNA, or modified RNA. The nucleic acids can also be extracellular or circulating nucleic acids, such as cfDNA or exRNA.

The target sequences can be any nucleotide sequence of interest that may be present in a sample. Typical target sequences include genes, transcription products (including alternatively spliced products), and biomarkers for diseases and other conditions.

Target sequences for detection also include nucleic acids that contain epigenetic modifications, such as methylation, which can be detected by performing additional steps or by performing steps in parallel, with or without the additional steps. For example, a sample can be divided into one aliquot for processing with bisulfite conversion (to convert cytosine to uracil, while leaving 5-methylcytosine intact) and another aliquot for processing without conversion, so that the results from the two aliquots can be compared to indicate the presence of 5-methylcytosine.

The number of target sequences to be amplified from a sample can vary from 1, 2, 5, 10, 20, 50, 100, 150, 200, 250, 300, 400, 500, 600, 700, 800, 900, 1000, 1200, 1400, 1500, 2000, or 5000 or more in multiplex reactions. The sequences can be selected based on published standards, recommended sets of markers or gathered by algorithmic means from databases, such as publicly available genomic and expression databases.

Each of the sequences to be amplified can vary in length from 1, 2, 5, 10, 20, 50, 100, 150, 200, 250, 300, 350, 400, 450, 500, 550, 600, 700, 800, 900, 1000, 1500, 2000, 5000, or 10,000 or more nucleotides in length. The longer targets can be amplified by staggered or tiling primers.

For a target sequence, multiple subsequences can be selected for amplification in the invention. For example, in FIG. 1, the full-length target sequence shown in the 1st Group reactions can have an amplicon that is essentially defined as the amplification product of the first and second primers. More specifically, the first primer has a predetermined portion for hybridization (SpF_1) and the second primer has a matching predetermined portion for hybridization (SpR_1), so that the amplification product will contain SpF_1, SpR_1 and the intervening target sequence that lies between predetermined portion SpF_1 and predetermined portion SpR_1. To the right, in the 2nd Group reactions, the sample target sequence can be amplified with a second pair of primers to yield an amplicon that has SpF_2, SpR_2 and the target sequence that lies between SpF_2 and SpR_2. Thus, a single target sequence can have several amplicons, each described here in terms of the pair of predetermined portions that are used to design the amplification primers for that amplicon. The number of amplicons to be amplified in the invention for a unique target sequence can be at least 1, 2, 3, 4, 5, 6, 7, 8, 9, 10, 12, 14, 16, 18, 20, 25, 30, 40 or 50 or more. Each of the amplicons can be described and later identified in terms of the pair of predetermined portions used to design the amplification primers for that amplicon.

The invention provides sets of primers to amplify the amplicons of target sequences. For a single amplicon, a first primer of the invention can have a forward tag sequence (such as TagF) and the upstream portion of the amplicon (such as SpF_1 or SpF_2) or their respective complements. A second primer can have a reverse tag sequence (such as TagR) and the downstream portion of the amplicon (such as SpR_1 or SpR_2) or their respective complements. The tag sequences can have sequences useful in downstream steps, such as landing sites for amplification and sequencing primers.

In some embodiments, the SpF and SpR portions of the primers can contain degenerate bases (synthesized by degrees of mixture of two, three, or four nucleoside phosphoramidites) or a universal base, such as inosine. The length of the degenerate sequence can be 0, 1, 2, 3, 4, 5, 6, 7, 8, 9, 10, 11, 12, 13, 14, 15, 16, 17, 18, 19, or 20 or more in one or more stretches of contiguous positions. The degenerate position(s) allow the primers to hybridize to variable regions of the target sequences or to amplify families of sequences, such as splice variants, using a compact set of primers.

The primers are typically DNA, but the invention provides primers with one or more non-naturally occurring base or bond. Modified nucleotides such as dideoxynucleotides, deoxyUridine (dU), 5-methylCytosine (5mC), 5-hydroxymethylCytosine (5hmC), 5-formylCytosine (5fC), 5-carboxylCytosine (5caC), and inosine can be used. Other modifications include modified bases such as 2,6-diaminopurine, 2-aminopurine, 2-flurobases, 5-bromoUracil, or 5-nitroindole. Other primers can have a modified sugar-phosphate backbone at one or more positions, such as a 3′-3′ or 5′-5′ linkage inversion, a locked nucleic acid (LNA), or a peptide nucleic acid (PNA) backbone.

The primers can also be modified with an exonuclease-resistant group at or adjacent to one end. Such modifications include an inverted nucleotide such as deoxythymidine (idT), a dideoxynucleotide such as dideoxythymidine (ddT or iddT), or 2′/3′-O-acetyation of the terminal nucleotide. One or more of the terminal nucleotides can be attached via one or more phosphorothioate bonds, LNA, or PNA backbones.

The primers of the invention can be labeled with a fluorescent moiety so they can be quantitated and detected by fluorescent means. A particularly useful technique is fluorescent resonance energy transfer (FRET) to provide relative distance information between labeled primers that are hybridized to potentially adjacent sequences.

The tag sequences (TagF or TagR) of the primers are generally an invariable or fixed sequence shared by a set of primers. This can allow subsequent hybridization or amplification steps using the same primers, such as the supplemental primers shown in FIG. 2.

If desired, any of the primers disclosed herein can incorporate one or more barcode sequences, for example an identifier 5′ to the sequence to be synthesized, so that the barcode becomes part of the amplified strand. The barcode sequence can be used to uniquely identify the sample in a multi-sample experiment, identify a group of reactions, or identify a particular target sequence. The barcode may incorporate redundancy or error-correction features. The barcodes can also be used to identify different lengths or degrees of degenerate sequences, or to distinguish between experiments or sample donors.

When a target sequence is best analyzed by amplifying different amplicons of the target, different barcodes can be used to identify the different amplicons of the same target sequence. Amplifying various sequences can present a problem, however, where the target is present (or potentially present) in widely varying numbers in a sample so there is a wide dynamic range. When libraries of multiple target sequences are to be obtained, conventional methods may amplify only the most numerous species, consuming the resources of the reaction so that less numerous species are not amplified in representative quantities, or not at all. Moreover, different regions of a target sequence may not be subject to primer amplification uniformly, so that the selection of different amplicon regions for amplification of a target can yield different or misleading results.

In an embodiment, various amplicons of a target sequence can be amplified in separate reactions. Where the reaction is multiplex amplification, the amplicons of multiple targets can be amplified in segregated groups of reactions. For example, in FIG. 1, a first group of reactions is shown on the left, a second group of reactions in the middle, and potential other groups of reactions to the right. The number of groups in the method of the invention can be more than 1, 2, 3, 4, 5, 6, 7, 8, 9, 10, 12, 14, 16, 18, 20, 25, 30, or 40 or more. Accordingly, the invention provides primers that can be used in a group reaction for multiple targets, as well as for multiple reactions for the individual targets. For example, a target can be amplified in more than 1, 2, 3, 4, 5, 6, 7, 8, 9, 10, 12, 14, 16, 18, 20, 25, 30, or 40 or more groups of reactions.

Nevertheless, a particular target need not to be amplified in all groups in the method. Some amplicons of a target may be amplified in one group reaction with other target sequences according to expected copy number. Other target amplifications can be segregated in reserved group reactions to avoid potential cross-hybridization between primers or other potentially unrepresentative or misinformative interactions between primers, target sequences and/or their amplicons. Potentially rare sequences to be amplified can be amplified with other rare sequences in separate groups so they are not out-amplified by moderately or highly abundant species, such as housekeeping genes.

The primers can be provided in the form of a cocktail for the desired set of targets, where at least one primer or primer pair is provided for each group.

The primers of the invention should be designed with certain constraints or priorities in mind when selecting among different possible amplicons for a target. The portion intended for hybridization to the target (such as SpF and SpR) can be 6, 7, 8, 9, 10, 11, 12, 13, 14, 15, 16, 17, 18, 19, 20, 22, 24, 25, 26, 27, 28, 29, 30, 32, 34, 36, 38, and 40 or more nucleotides in length, taking into consideration the number of G and C bases and their proximity to primer ends on predicted melting temperature. The sequence of the primer can be selected or prioritized to avoid the potential for cross-hybridization with other primers present in the same reaction. For example, a predetermined portion of an amplicon can be selected to avoid self-hybridization (such as hairpins) or cross-hybridization with other predetermined portions to be used in a reaction of a group (such as primer dimers). The predetermined portions can also be selected to avoid hybridization with sequences selected from the group consisting of sequences expected in a gDNA sample, sequences containing known SNPs, known repetitive sequences, and known nontranscribed sequences. These considerations also apply to the tag portions of the primers, as well as consideration of the tag portions when adjacent to the predetermined portions.

The primers for two amplicons of a target can be selected so that the predetermined portion of one overlaps with the predetermined portion of the other. This can result in amplicons that share a relatively long stretch of identical sequence, but whose primers (and group reactions) can be identified by the offset of the starting or ending sequence. Preferred offsets include 1, 2, 3, 4, 5, 6, 7, 8, 9, 10, 11, 12, 13, 14, 15, 16, 17, 18, 19, 20, 22, 24, 26, 28, and 30 or more bases between comparable primers (e.g. offsets between SpF_1 and SpF_2, or offsets between SpR_1 and SpR_2).

The primers can also be selected so that a single forward primer can be used with more than one reverse primer, or vice versa. The pairs of primers to be used in different groups can also be provided in numbers that normalize for the potential range of abundance of targets present in a sample, and their abundance relative to other targets that may be present. These calculations may be based on various sources, including available data about the target, empirical testing of the sample or similar samples, or expected levels from functional assays. Thus, the number of primers in a reaction can be tuned for balanced amplification of a target in a first group relative to other groups. The ratio of primers between different groups for the same target can vary between about 5%, 10%, 20%, 25%, 33%, 50%, 66%, 75%, 80%, about equal amounts, 120%, 133%, 150%, 175%, 2×, 2.5×, 3×, 4×, 5×, and 10× relative to each other, including ranges of these ratios. In addition, each of the first, second, and optional universal primers can be provided in different ratios relative to each other, such as 5%, 10%, 20%, 25%, 33%, 50%, 66%, 75%, 80%, about equal amounts, 120%, 133%, 150%, 175%, 2×, 2.5×, 3×, 4×, 5×, and 10×, including ranges of these ratios.

Another useful embodiment involves addition of neutralization oligos to groups of reactions, where a particular target species is expected to be high and may consume a large portion of reaction resources. Such oligos can have a sequence identical or complementary to a predetermined portion to hinder the hybridization of primers or displace primers from the predetermined portions, blocking amplification from taking place. When cocktails of primers have been prepared for sets of targets or groups as stock solutions, the addition of sets of neutralization oligos can provide a convenient layer of customization to amplification reactions, according to the intended purpose.

### Hybridization

As illustrated in FIG. 1, the primers of the invention can be contacted with a target sequence, resulting in a reaction mixture. The components of the mixture can be allowed to hybridize according to conditions that can be selected by the skilled artisan to allow and optimize for hybridization between the polynucleotides with the desired degree of specificity or mismatches. Such conditions will vary with the lengths and compositions of sequences present in the hybridization reaction, the nature of any modifications, as well as conditions such as the concentrations of the polynucleotides and ionic strength. Particular reaction io conditions include temperatures include 25°, 30°, 32.5°, 35°, 37.5°, 40°, 42.5°, 45°, 47.5°, 50°, 52.5°, 55°, 57.5°, 60°, 62.5°, 65°, 67.5°, 70°, 72.5°, 75°, 77.5°, 80°, 82.5°, 85°, 87.5°, 90°, 95°, 100°, 105°, 110°, and/or 120° C. including combinations of such temperatures for various times, including ramping periods between temperatures. Ions such as Li⁺, Na⁺, K⁺, Ca²⁺, Mg²⁺ and/or Mn²⁺ can also each be present from 0, 1, 2, 5, 10, 20, 50, 100, 200, and 500 mM or more, and the inclusion of such ions can affect the selection of the other hybridization conditions.

Hybridization is also affected by steric crowding components such as branched polysaccharides, glycerol, and polyethylene glycols (where useful MWs can vary from 100, 200, 400, 800, 1000, 2000, 4000, 6000, 8000, 10,000, 20,000, or higher, in linear, multi-armed, branched, and functionalized versions). Further additives can be present in the hybridization (and subsequent) reactions, such as DMSO, non-ionic detergents, betaine, dithiothreitol, ethylene glycol, 1,2-propanediol, formamide, tetramethyl ammonium chloride (TMAC), and/or proteins such as bovine serum albumin (BSA), according to the desired specificity, stringency, or hybridization conditions.

After hybridization, excess components can be removed by various conventional steps, such as attachment to a solid phase and washing, centrifugation of solutes away from precipitates, and microfluidic separation.

### First Amplification (PCR1)

Many amplification methods and instruments are commercially available, and the amplification enzymes (such as Pfu, Taq, KOD and their commercial variants such as Phusion) and reaction conditions can be selected and tailored to the particular platform. The polymerase selected for amplification can be Bst DNA polymerase, large fragment; Bsu DNA polymerase, large fragment; Vent DNA polymerase; E. coli DNA polymerase I; M-MuLV reverse transcriptase; phi29 DNA polymerase, etc.

If desired, the enzyme used in amplification steps can have a hot-start feature that uses an antibody interaction, a chemical modification or an aptamer to allow reaction set-up at room temperature or to reduce non-specific amplification.

As a result, the invention provides a library of amplicons of a group obtained by performing the first amplification step. When barcodes are present in the amplification primers, the library can contain one or more barcodes that can carry the intended information. Matching two or more barcodes in an amplicon can be used to confirm the intended amplification product was obtained, or to detect when unintended amplification products are produced, such as when a primer intended to amplify one target amplifies a different target (misamplification). Thus, the barcodes serve as quality control indicators for the primer design and amplification process. In other embodiments, the presence of matching sequences of the predetermined portions can serve the role of barcodes to identify intended or unintended amplicons. For example, when reads are produced from a set of primers that combine unexpected combinations of barcodes and/or predetermined regions, the misamplification products can be used to trouble-shoot and improve the primer designs, manually or informatically.

### Pooling & Supplemental Amplification (PCR2)

The invention provides the step of pooling the products of separate group reactions to provide a pooled library of amplicons. If desired, the pooled library can be amplified a second time in an optional supplemental step with a supplemental set of primers, as exemplified in FIG. 2. In the illustrated embodiment, the design in FIG. 1 allows all species of the pooled library to be amplified with a primer having TagF and another primer. One or both primers may contain a barcode, as discussed above. Preferably the primers contain sequences that allow the products of the supplemental amplification to be ready for sequencing on conventionally available instruments and workflows. For example, the supplemental primers can have sequences that enable attachment to solid phases for further reactions such as purification, washing, or additional amplification steps. Thus, the invention provides a sequencing-ready library of amplicons obtained by performing the segregated grouping, multiplexed method.

The invention further provides reagent kits for performing the invention that include the primer cocktails and optional neutralization oligos. The kits can also include primers suitable for the supplemental amplification.

The end user may use polymerases and other components obtained elsewhere, or the kits provided can also include enzymes for amplification, such as polymerases for performing isothermal amplification or PCR. The kits can further provide reaction buffers for the enzymes in the kit or buffer components to be added to reactions suitable for the enzymes. The kits can further include components to optimize the hybridization step and to improve the efficiency of the amplification steps, including the steric crowding components and other reaction additives provided above.

Although the workflows described herein are intended to provide libraries ready for sequencing, other sequence-detection methods can be used, such as qPCR, end point PCR, enzymatic, optical, or labeling for detection on an array or other molecule detection.

### Bioinformatic Methods for Optimizing Primers for Multiplex Amplification

The present invention also provides bioinformatic methods for optimizing the design of primers. As discussed above, the forward tag sequence and reverse tag sequence should serve as sequences that become part of the amplicon without interfering with other reactions. If the tag sequences self- or cross-hybridize, or otherwise cause undesirable or intended interactions with other reaction components, then the absence or malformation of amplicons becomes informative. On the other hand, when the specific sequences (SpF_x and SpR_x) are not optimally selected at first (e.g., primers containing common single nucleotide polymorphisms), it could result in allele drop-out or no amplification of the target. When the primer sequences are designed by algorithms or heuristic methods, the information can be used to provide feedback to improve the primer design by driving selection. The detection of malformed amplicons can also be analyzed topologically to troubleshoot for likely causes for the undesired amplification, for example when a primer hybridizes to a sequence that occurs multiple times in a target sequence within an amplifiable distance. The information from such analysis can then be used to prepare a subsequent set of primers for use with the same or modified groups for a subsequent amplification, leading to further amplicon analysis, refinement of primers, and so on.

In addition, the analysis of amplicons may show that certain target sequences are under- or overamplified by primers in one reaction group or another. For example, an amplicon of a target sequence may be difficult to amplify or more easily amplified due to differences in hybridization properties (such as length or CG %) of the predetermined regions for hybridization to primers. The differences can be compensated for by improving the primers or primer sets, such as by tuning (increasing or decreasing) the concentration of primers in that group reaction. The location of the predetermined regions appearing in a primer can also be shifted to include or exclude more certain sequence motifs such as runs of repeated bases or dinucleotides, or the length can be increased or decreased. The predetermined regions in the primers can further be modified with degenerate or universal bases. The primer amplification of amplicons that are under- or overamplified one group reaction can also be adjusted by moving the primers to another group reaction. This can be desirable when primers originally in one group reaction interact with other primers that group reaction.

Among amplification products, the percentage of undesired amplicons can therefore be decreased from 60, 65, 70, 75, or 80% or greater to less than 25%, 20%, 18%, 16%, 15%, 14%, 12%, 10%, 8%, 6%, 4%, or fewer. The reduction and prevention of such amplicons reduces waste in reaction, sequencing and computing resources, and results in a significant reduction in the cost per sample analyzed.

Another consideration for the iterative primer design of the invention is to favor predetermined portions that have overlapping sequences among primers for the same target or to have offsets of more than a minimum number of bases to facilitate analysis for feedback. Other modifications to the primer designs based on feedback can be to introduce modified, degenerate or universal bases. The improved primer design can also incorporate the step of adding neutralization oligos, and critically, such oligos can be subjected to similar iterative improvements. Accordingly, the invention provides cocktails of improved primers and libraries of amplicons obtained by using the improved primers.

### EXAMPLES

### Example 1: Multiplex Amplification Kit with Reduced Misamplification

A version of the multiplex amplification kit contains reagents to amplify over 1000 amplicons from over 500 genomic targets. Among usable reads, the average coverage for each genomic locus was >1000×. Using the methods of the invention provided herein to optimize primer design, the nonspecific amplification rate was reduced from >80% to <15%.

### Example 2: Bioinformatic Optimization of Primers

A set of primers are prepared to amplify at least two different amplicons (_1, and _2, sometimes _3, _4, or _5) each of 5 target sequences, AZ_11004, AZ_11071, AZ_10106, AZ_10082, and AZ_10666, in separate groups of reactions. For example, the forward primer for the first amplicon of AZ_11004 has the predetermined region AZ_11004 1F (as well as a TagF sequence).

| | |
|---|---|
| AZ_11004: amplicon_1: forward primer has sequence | AZ_11004_1F |
| reverse primer has sequence | AZ_11004_1R |
| amplicon_2: forward primer has sequence | AZ_11004_2F |
| reverse primer has sequence | AZ_11004_2R |
| AZ_11071: amplicon_1: forward primer has sequence | AZ_11071_1F |
| reverse primer has sequence | AZ_11071_1R |
| amplicon_2: forward primer has sequence | AZ_11071_2F |
| reverse primer has sequence | AZ_11071_2R |

Similarly, the primers for the other three targets include
AZ_10106: AZ_10106_1F, AZ_10106_1R, AZ_10106_3F, AZ_10106_3R, AZ_10082: AZ_10082_2F, AZ_10082_2R, AZ_10082_3F, AZ_10082_3R, AZ_10082_4F, AZ_10082_4R (for a third amplicon AZ_10082).
AZ_10666: AZ_10666_1F, AZ_10666_1R, AZ_10666_2F, AZ_10666_2R, AZ_10666_4F, AZ_10666_4R (for a third amplicon of AZ_10666), AZ_10666_5F, AZ_10666_5R (for a fourth amplicon of AZ_10666).

The expected amplification product of the pair of primers having AZ_11004_1F and AZ_11004_1R is an AZ_11004_1 amplicon, which should contain the sequences AZ_11004_1F, an intervening sequence of the target sequence, and AZ_11004_1R. Other expected amplification products include those with AZ_11004_2F and AZ 11004_2R; AZ_11071_1F and AZ_11071_1R; and AZ_10666_4F and AZ_10666_4R.

However, the detection of amplicons having the following sequences would be unexpected and suggest some kind of misamplification events, such as during PCR1:
AZ_11004_1F and AZ_11071_1R (misamplification in group 1 reaction)
AZ_11071_2F and AZ_10082_2R (misamplification in group 2 reaction)
AZ_10082_4F and AZ_10666_4R, (misamplification in group 4 reaction)

Also, detection of amplicons having the following unintended sequences would also be unexpected:
AZ_11004_1F and AZ_11004_2R (suggesting cross-contamination of groups 1 and 2)
AZ_11004_1F and AZ_10666_1F (suggesting hybridization of a primer intended for one target sequence to a similar target sequence in the same group)

Other malformed amplicons can be analyzed to troubleshoot for likely causes for the undesired amplification, for example hybridization to unintended regions during PCR2.

Moreover, where an expected amplicon is amplified in unrepresentatively high numbers, the amplicon can be undesired because it consumes an undesired amount of reaction resources for an intended purpose.

Accordingly, upon such analysis of the amplicons, an improved set of primers can be prepared to reassign the role of an original primer with a substitute primer that has a different predetermined region, such as a region that is offset from the original region, or selected to be from a different predetermined region of the desired target sequence. An improved set of primers may also include selected neutralization oligos to reduce the number of undesired amplicons. The improved primer set can be used to further amplify target sequences in a sample, for further analysis of the resulting amplicons, and further optimization of the predetermined portions to prepare further improved primer sets by iterative feedback optimization.

The headings provided above are intended only to facilitate navigation within the document and should not be used to characterize the meaning of one portion of text compared to another. Skilled artisans will appreciate that additional embodiments are within the scope of the invention. The invention is defined only by the following claims; limitations from the specification or its examples should not be imported into the claims.

I claim:

1. A method for obtaining a library of multiple amplicons of one or more target sequences in a sample, performed in at least two segregated groups of reactions, comprising the steps of
(1) for the reaction of the first group to amplify the first amplicon of the target, wherein the first amplicon has a predetermined portion at an upstream end, and a predetermined portion at a downstream end,
(a) contacting the sample with
a first primer comprising a forward tag sequence and the upstream portion of the amplicon (or its complement);
a second primer comprising a reverse tag sequence and the downstream portion of the amplicon (or its complement);
an optional universal primer comprising the forward or the reverse tag sequence and an optional barcode;
(b) amplifying the first amplicon in a reaction for the first group;
(2) for the reaction of the second group to amplify the second amplicon of the target, wherein the second amplicon has a predetermined portion at an upstream end, and a predetermined portion at a downstream end,
(a) contacting the sample with
a first primer comprising the forward tag sequence and the upstream portion of the region (or its complement);
a second primer comprising the reverse tag sequence and the downstream portion of the region (or its complement);
an optional universal primer comprising the forward or reverse tag sequence and an optional barcode;
(b) amplifying the second amplicon in a segregated reaction for the second group;
(3) pooling the amplicons from the segregated reactions;
(4) optionally amplifying the pooled amplicons; and
(5) optionally adding a secondary barcode to the pooled amplicons;
thereby obtaining a library of different amplicons of targets that were amplified in segregated groups of reactions.

2. The method of claim 1, wherein the number of groups is greater than 3, 4, 5, 6, 7, 8, 9, 10, 12, 14, 16, 18, 20, 25, 30, or 40.

3. The method of claim 1, wherein a target sequence is amplified in fewer than all groups.

4. The method of claim 1, wherein the predetermined portions of an amplicon are selected to avoid cross-hybridization with other predetermined portions to be used in a reaction of a group, to avoid self-hybridization, or to avoid hybridization with sequences selected from the group consisting of sequences expected in a gDNA sample, sequences containing known SNPs, known repetitive sequences, and known nontranscribed sequences.

5. The method of claim 1, wherein the number of first and second primers in a reaction is tuned for balanced amplification of a target in a first group relative to other groups.

6. A cocktail of primers for performing steps (1) and (2) of claim 1, having at least one primer per group.

7. The cocktail of claim 6, wherein a primer is modified with a non-naturally occurring base or bond, an exonuclease-resistant group, or a fluorescent moiety.

8. The cocktail of claim 6, wherein the number of groups is at least three.

9. The cocktail of claim 6, wherein the number of primers provided for a first group reaction is tuned relative to the number of primers provided for a second group reaction.

10. The cocktail of claim 6, further comprising universal primers, and a supplemental set of amplification primers, or reaction components for steps (1) and (2).

11. A reaction mixture of a group obtained by performing step (1)(a) of the method of claim 1.

12. A library of amplicons of a group obtained by performing step (1), and optionally steps (2), (3), (4), or (5) of the method of claim 1.

13. A method for selecting predetermined portions to amplify at least two amplicons of target sequences, comprising:
(A) preparing primer sets for at least two segregated groups of reactions, comprising the steps of
(1) for the reaction of the first group to amplify the first amplicon of the target, wherein the first amplicon has a predetermined portion at an upstream end, and a predetermined portion at a downstream end,
(a) contacting the sample with
a first primer comprising a forward tag sequence and the upstream portion of the amplicon (or its complement);
a second primer comprising a reverse tag sequence and the downstream portion of the amplicon (or its complement);
an optional universal primer comprising the forward or the reverse tag sequence and an optional barcode; and
(b) amplifying the first amplicon in a reaction for the first group;
(2) for the reaction of the second group to amplify the second amplicon of the target, wherein the second amplicon has a predetermined portion at an upstream end, and a predetermined portion at a downstream end,
(a) contacting the sample with
a first primer comprising the forward tag sequence and the upstream portion of the region (or its complement);
a second primer comprising the reverse tag sequence and the downstream portion of the region (or its complement);
an optional universal primer comprising the forward or reverse tag sequence and an optional barcode;
(b) amplifying the second amplicon in a segregated reaction for the second group;
(3) pooling the amplicons from the segregated reactions;
(4) optionally amplifying the pooled amplicons; and
(5) optionally adding a secondary barcode to the pooled amplicons;
(B) analyzing the library of amplicons for unexpected or undesired amplicons; and
(C) preparing improved primer sets, having at least one different predetermined portion compared to the primer sets prepared in step (A).

14. The method of claim 13, wherein step (C) comprises tuning the number of first and second primers in a reaction.

15. The method of claim 13, wherein step (C) comprises moving primers from one group in step (A) to another group.

16. The method of claim 13, wherein the predetermined portions of two amplicons of a target sequence are offset by at least 3, 4, 5, 6, 7, 8, 9, 10, 12, 14, 16, 18, or 20 bases.

17. The method of claim 13, further comprising steps (B) to (C) performed iteratively.

18. A cocktail of improved primers obtained by performing the method of claim 13.

19. Reaction mixtures obtained by performing the method of claim 13 to obtain improved primers and further performing steps (1)(a) and (2)(a) with the improved primers.

20. A library of amplicons of a group obtained by further performing steps (1)(b) and (2)(b) on the reaction mixtures of claim 19.
